(19) United States
(12) Patent Application Publication
(10) Pub. No.: US 2022/0412876 A1
(43) Pub. Date: Dec. 29, 2022

(54) SULFIDATION DETECTION SENSOR

(71) Applicant: KOA CORPORATION, Ina-shi (JP)

(72) Inventor: Taro KIMURA, Ina-shi (JP)

(73) Assignee: KOA CORPORATION, Ina-shi (JP)

(21) Appl. No.: 17/835,595

(22) Filed: Jun. 8, 2022

(30) Foreign Application Priority Data
Jun. 25, 2021 (JP) 2021-105797

(51) Int. Cl. G01N 17/02; G01N 17/04

(52) U.S. Cl. CPC G01N 17/02; G01N 17/04; G01N 27/4045

(57) ABSTRACT

A sulfidation detection sensor includes a rectangular parallelepiped insulating substrate, a resistor formed to adhere closely to a surface of the insulating substrate, a sulfidation detection conductor formed to adhere closely to a surface of the resistor, a protective layer that is impermeable to sulfide gas and formed to cover a portion of the sulfidation detection conductor, and a pair of electrode portions formed at both ends of the insulating substrate and connected to the resistor and the sulfidation detection conductor. The sulfidation detection conductor is made of metal having a resistance value less than that of the resistor, and includes an exposed portion exposed to the outside without being covered with the protective layer.

### TECHNICAL FIELD

The present invention relates to a sulfidation detection sensor for detecting a cumulative amount of sulfide in a corrosive environment.

### BACKGROUND ART

As an internal electrode of an electronic component such as a chip resistor, generally, an Ag (silver) based electrode material having a low specific resistance is used. However, silver is converted into silver sulfide when being exposed to sulfide gas and the silver sulfide is an insulator, which may cause malfunction such as disconnection of an electronic component. In recent years, measures against sulfidation, such as forming an electrode that hardly gets sulfurized by adding Pd (palladium) and Au (gold) to Ag, or forming the electrode into a structure that prevents sulfide gas from reaching the electrode have been taken.

However, even when such measures against sulfidation are taken for an electronic component, in the case where the electronic component is exposed to sulfide gas for a long time or exposed to high-concentration sulfide gas, disconnection cannot be prevented completely. Accordingly, it is necessary to detect the disconnection in advance to prevent failure from occurring at an unexpected timing.

With this regard, as described in Patent Literature 1, there has been proposed a sulfidation detection sensor capable of detecting the degree of cumulative sulfide in an electronic component to detect a risk of failure such as disconnection which occurs in an electronic component due to sulfidation. By way of example, a sulfidation detection sensor disclosed in Patent Literature 1 is designed such that a sulfidation detection conductor mainly made of Ag is provided on an insulating substrate, a transparent protective film which is permeable to sulfide gas is provided so as to cover the sulfidation detection conductor, and end face electrodes connected to the sulfidation detection conductor are provided, respectively, at both side end portions of the insulating substrate.

When the sulfidation detection sensor designed as described above is mounted on a circuit board together with other electronic components and then the circuit board is used in an atmosphere containing sulfide gas, the other electronic components get sulfurized over time, and the sulfide gas permeates through the protective film of the sulfidation detection sensor and comes into contact with the sulfidation detection body. This causes the sulfidation detection body to tarnish gradually in accordance with the concentration of sulfide gas and an elapsed time. Furthermore, as sulfidation proceeds, silver that forms the sulfidation detection body gets converted to silver sulfide, whereby a resistance value of the sulfidation detection sensor gradually increases, and eventually disconnection occurs. Accordingly, visually observing change in color of tarnish on the sulfidation detection body through the protective film, irradiating a light onto the upper surface of the sulfidation detection sensor to detect a reflected light from the sulfidation detection body, or detecting change in a resistance value of the sulfidation detection body enables detection of the degree of sulfidation.

### CITATION LIST

### Patent Literature

Patent Literature 1: JP-A-2009-250611

### SUMMARY OF INVENTION

### Technical Problem

However, since color of tarnish on the sulfidation detection conductor, which is caused by sulfide gas, changes delicately, it is difficult to accurately detect the degree of sulfidation based on visual observation by an operator. Furthermore, in the case of detecting the degree of sulfidation based on the reflected light from the sulfidation detection conductor, there is a problem that a large-scale facility for this detection is separately required.

Still further, in the case of detecting the change in a resistance value of the sulfidation detection conductor, since the sulfidation detection conductor is a conductor mainly composed of Ag or the like having a low specific resistance, the amount of change in the resistance value in the time during which the sulfidation detection conductor starts to get sulfurized to eventually gets disconnected is extremely small. Accordingly, it is difficult to accurately detect the degree of sulfidation based on the change in a resistance value of the sulfidation detection conductor during the time above.

The present invention has been made in view of the circumstances above of the prior art, and an object of the present invention is to provide a sulfidation detection sensor capable of accurately and easily detecting the degree of sulfidation.

### Solution to Problem

In order to achieve the object described above, the present invention provides A sulfidation detection sensor, comprising: a rectangular parallelepiped insulating substrate; a resistor that is provided on a main surface of the insulating substrate; a sulfidation detection conductor that is provided on the resistor and to be sulfurized by sulfide gas; a protective layer that is impermeable to the sulfide gas, and provided so as to cover a portion of the sulfidation detection conductor; and a pair of electrode portions that is provided at both ends of the insulating substrate, respectively, and connected to the resistor and the sulfidation detection conductor, wherein the sulfidation detection conductor is made of metal having a resistance value smaller than that of the resistor, and includes an exposed portion exposed to an outside without being covered with the protective layer.

In the sulfidation detection sensor designed as described above, the pair of electrode portions is always conductive to each other through the resistor, and when the sulfidation detection sensor is exposed in an atmosphere containing sulfide gas and thus sulfidation proceeds, the sulfidation detection conductor provided on the resistor starts to get sulfurized, from the exposed portion exposed to the outside without being covered with the protective layer, and then toward the inside thereof covered with the protective layer. This causes change in the current path extending between the pair of electrode portions in accordance with the degree of sulfidation of the sulfidation detection conductor. As a result, the resistance value of the resistor continuously changes in accordance with the degree of sulfidation of the sulfidation detection conductor, thereby enabling accurate and easy detection of the degree of sulfidation.

In the sulfidation detection sensor having the structure as described above, the sulfidation detection conductor may be formed so as to cover the entire surface of the resistor. On the other hand, in the case where the resistor includes an adjustment region which is not covered with the sulfidation detection conductor, the adjustment region is provided with a trimming groove for adjusting a resistance value, and the adjustment region is covered with a part of the protective layer, it is possible to, not only increase an initial resistance value of the resistor by the trimming groove, but also realize a sulfidation detection sensor which excels in the temperature characteristics (TCR).

In the sulfidation detection sensor having the structure as described above, the sulfidation detection conductor may be formed of a single material. On the other hand, in the case where the sulfidation detection conductor is composed of a first sulfidation detection conductor and a second sulfidation detection conductor which are made of different materials with different selectivity of gas, and each of the first sulfidation detection conductor and the second sulfidation detection conductor includes the exposed portion, it is possible to reliably detect the degree of sulfidation regardless of the type of sulfide gas contained in an atmosphere during use.

That is, the reactivity of sulfide gas differs depending on the type of metal forming the sulfidation detection conductor, for example, silver (Ag) easily reacts with hydrogen sulfide (H₂S) but hardly reacts with sulfur dioxide (SO₂) while nickel (Ni) easily reacts with sulfur dioxide (SO₂) but hardly reacts with hydrogen sulfide (H₂S), and accordingly, in the case of forming one of the first sulfidation detection conductor and the second sulfidation detection conductor with Ag while forming the other with Ni, it is possible to realize a sulfidation detection sensor which is applicable for different types of sulfide gases. Note that, since copper (Cu) is a material that is easy to react with both hydrogen sulfide (H₂S) and sulfur dioxide (SO₂), solely using copper (Cu) can realize a sulfidation detection sensor which is applicable for multiple types. However, like the combination of Ag material and Ni material as described above, combining copper (Cu) with a material with different selectivity of gas and high responsibility to the target sulfide gas can improve the accuracy of detection more than the case of forming a sulfidation detection body solely using copper (Cu).

In this case, it is preferable that the resistor includes an exposed region which is not covered with the first sulfidation detection conductor and the second sulfidation detection conductor, an intermediate protective layer is formed on the exposed region, and the exposed portion of the first sulfidation detection conductor and the exposed portion of the second sulfidation detection conductor are disposed at positions interposing the intermediate protective layer therebetween.

Furthermore, in the sulfidation detection sensor having the structure as described above, the resistor and the sulfidation detection conductor may be metal glaze thick films formed by screen-printing or the like. On the other hand, in the case where each of the resistor and the sulfidation detection conductor is made of a metal film formed as a thin film by sputtering or the like, it is possible to eliminate variations in the film thicknesses of the resistor and sulfidation detection conductor, and thus enhance the accuracy of detection.

In this case, in the case where the insulating substrate is made of an alumina substrate, and the resistor is a metal film of Ni—Cr formed by sputtering on a surface of the alumina substrate, preferably, it is possible to increase the bonding force between the resistor and the alumina substrate due to Cr in the metal film, and also, due to Ni therein, increase the adhesion between the resistor and the sulfidation detection conductor (Ag, Cu, Ni or the like).

Still further, in the sulfidation detection sensor designed as described above, it is preferable that the protective layer is composed of an undercoat layer made of a glass material formed on the sulfidation detection conductor and an overcoat layer made of a resin material formed on the undercoat layer, and each of the electrode portions covers an end of the sulfidation detection conductor and adheres closely to the overcoat layer. With this design, the adhesion between the electrode portions and the overcoat layer made of a resin material can be improved, and accordingly, it is possible to suppress sulfidation of end portions of the sulfidation detection conductor covered with the electrode portions. In addition, the undercoat layer through which sulfide gas cannot permeate is provided under the overcoat layer made of a resin material, and accordingly, it is possible to prevent a portion of the sulfidation detection conductor which is covered with the protective layer from reacting with the sulfide gas which has permeated through the overcoat layer and thus getting sulfurized.

### Advantageous Effects of Invention

According to the sulfidation detection sensor of the present invention, it is possible to accurately and easily detect the degree of sulfidation.

### BRIEF DESCRIPTION OF DRAWINGS

FIG. 1 is a plan view of a sulfidation detection sensor according to the first embodiment.

FIG. 2 is a cross-sectional view taken along the line II-II illustrated in FIG. 1.

Each of FIG. 3A to FIG. 3F is a plan view illustrating producing processes of the sulfidation detection sensor according to the first embodiment.

Each of FIG. 4A to FIG. 4F is a cross-sectional view illustrating the producing processes of the sulfidation detection sensor according to the first embodiment.

Each of FIG. 5A and FIG. 5B illustrates change in a current path in the sulfidation detection sensor according to the first embodiment.

FIG. 6 illustrates the relation between an elapsed time and a resistance value in the sulfidation detection sensor according to the first embodiment.

FIG. 7 is a plan view of a sulfidation detection sensor according to the second embodiment.

FIG. 8 is a cross-sectional view taken along the line VIII-VIII illustrated in FIG. 7.

FIG. 9 is a plan view of a sulfidation detection sensor according to the third embodiment.

FIG. 10 is a cross-sectional view taken along the line X-X illustrated in FIG. 9.

FIG. 11 is a plan view of a sulfidation detection sensor according to the fourth embodiment.

FIG. 12 is a cross-sectional view taken along the line XII-XII illustrated in FIG. 11.

### DESCRIPTION OF EMBODIMENTS

Hereinafter, embodiments of the present invention will be described with reference to the drawings.

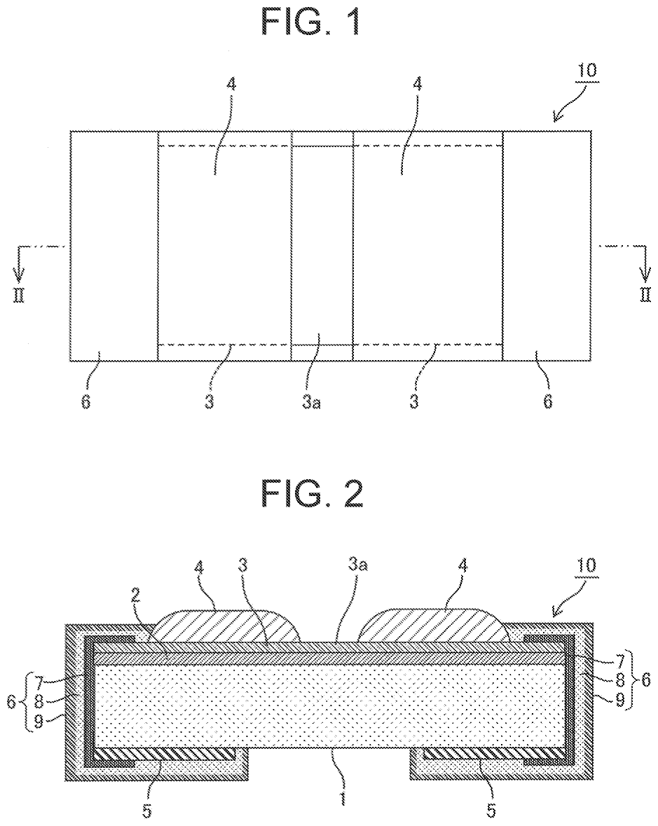

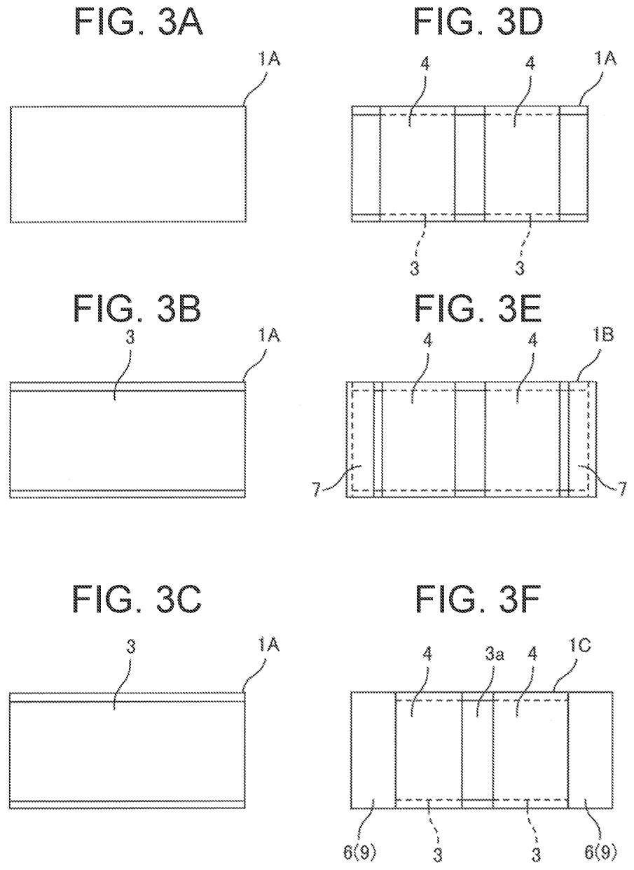

FIG. 1 is a plan view of a sulfidation detection sensor according to a first embodiment of the present invention, and FIG. 2 is a cross-sectional view taken along the line II-II illustrated in FIG. 1. As illustrated in FIG. 1 and FIG. 2, a sulfidation detection sensor 10 according to the first embodiment mainly includes a rectangular parallelepiped insulating substrate 1, a resistor 2 formed to adhere closely to a surface of the insulating substrate 1, a sulfidation detection conductor 3 formed to adhere closely to a surface of the resistor 2, sulfide gas-impermeable protective layers 4 formed to cover a part of the sulfidation detection sensor, a pair of back electrodes formed at both ends in the longitudinal direction of a back surface of the insulating substrate 1, and a pair of electrode portions 6 formed at both ends in the longitudinal direction of the insulating substrate 1.

The insulating substrate 1 is obtained by dividing a large-sized substrate, which will be described later, along vertical and horizontal division grooves into multiple pieces. The large-sized substrate is an alumina substrate containing alumina as a main component (purity: 96%).

The resistor 2 is made of a metal film of Ni—Cr, which is formed as a thin-film by sputtering, vapor deposition, or the like on the surface of the insulating substrate (alumina substrate). The resistor 2 is formed in a rectangular shape so as to extend over the entire surface of the insulating substrate 1, and both ends of the resistor 2 are connected to the pair of electrode portions 6, respectively.

The sulfidation detection conductor 3 is made of a metal film such as Cu, Ag, or Ni, which is formed as a thin film by sputtering, vapor deposition, or the like on the surface of the resistor 2. A resistance value of such a metal film is sufficiently smaller than a resistance value of the metal film forming the resistor 2 (for example, several KΩ in the case of the resistor, several tens mΩ in the case of the sulfidation detection conductor). The sulfidation detection conductor 3 is formed in a rectangular shape so as to extend over the entire surface of the resistor 2, and both ends of the sulfidation detection conductor 3 are also connected to the pair of electrode portions 6, respectively.

Each of the protective layers 4 is formed of an insulating material which is impermeable to sulfide gas, and has, for example, a double layer structure in which an overcoat layer made of a resin material is laminated on an undercoat layer made of a glass material. The protective layers 4 are formed at two positions excluding the central portion and both ends of the sulfidation detection conductor 3, respectively, and thus the central portion of the sulfidation detection conductor 3, which is not covered with the protective layers 4, is an exposed portion 3a exposed to the outside.

Each of the back electrodes 5 is made of a metal film of Cr—Cu or Cr—Ni—Cu, which is formed as a thin film by sputtering on the back surface of the insulating substrate (alumina substrate) 1. The pair of back electrodes 5 is formed at both ends in the longitudinal direction of the back surface of the insulating substrate 1, respectively. Note that, instead of the back electrodes 5 formed as a thin film, a thick film may be formed, and in such a case, the thick film can be formed by screen-printing an Ag paste or a Cu paste and drying and sintering the paste.

Each of the electrode portions 6 includes an end face electrode 7, an intermediate electrode 8, and an external electrode 9. The end face electrode 7 has a U-shaped cross-section to make the end of the sulfidation detection conductor 3 exposed from the protective layer 4 conductive with corresponding one of the back electrodes 5. The intermediate electrode 8 and the external electrode 9 are sequentially formed so as to cover the end face electrode 7. The end face electrode 7 is obtained by sputtering Ni/Cr on an end face of the insulating substrate 1. The intermediate electrode 8 is a Ni plating layer formed by electrolytic plating, and the external electrode 9 is a Sn plating layer formed by electrolytic plating.

Next, producing processes of the sulfidation detection sensor 10 designed as described above will be explained with reference to each of FIG. 3A to FIG. 3F and each of FIG. 4A to FIG. 4F. Each of FIG. 3A to FIG. 3F is a plan view illustrating a surface of a large-sized substrate used in these producing processes. Each of FIG. 4A to FIG. 4F is a cross-sectional view of one chip, which is taken along a central portion in the longitudinal direction of each of FIG. 3A to FIG. 3F.

As illustrated in FIG. 3A and FIG. 4A, firstly, a large-sized substrate 1A from which multiple pieces of insulating substrates 1 are obtained is prepared. In the large-sized substrate 1A, primary division groove and secondary division groove are provided in advance to form a grid pattern, and each one of the grids divided by the primary division groove and the secondary division groove serves as a single chip region. Each of FIG. 3A to FIG. 3F and each of FIG. 4A to FIG. 4F illustrate the large-sized substrate 1A corresponding to a single chip region as a representative example, but practically, each process which will be described below is collectively performed with respect to the large-sized substrate 1A corresponding to multiple pieces of chip regions.

That is, after sputtering Ni—Cr on the surface of the large-sized substrate 1A, a process of sputtering Cu or the like thereon is performed to form a metal film having a double layer structure. Thereafter, by patterning these metal films into a rectangular shape using photolithography, as illustrated in FIG. 3B and FIG. 4B, a process of forming the resistor 2 adhering closely to the surface of the large-sized substrate 1A and the sulfidation detection conductor 3 adhering closely to the surface of the resistor 2 is performed.

Next, by sputtering Cr—Cu or Cr—Ni—Cu on the back surface of the large-sized substrate 1A from above a mask (mask sputtering), as illustrated in FIG. 3C and FIG. 4C, a process of forming the back electrodes 5 oppositely disposed on the back surface of the large-sized substrate 1A with a predetermined distance therebetween is performed.

Next, after forming an SiO₂ film by the chemical vapor deposition (CVD) method or sputtering from the surface side of the large-sized substrate 1A, or forming the undercoat layer by screen-printing a glass paste and then drying and sintering the paste, a process of screen-printing an epoxy resin or a phenolic resin on the undercoat layer and heating and curing the paste is performed to form the overcoat layer. Thus, as illustrated in FIG. 3D and FIG. 4D, the protective layers 4 each of which covers a portion of the sulfidation detection conductor 3, excluding the central portion and both the ends thereof, can be obtained.

Next, after primarily dividing the large-sized substrate 1A along the primary division groove to obtain a strip-shaped substrate 1B, by sputtering Ni/Cr on each of the divided faces of the strip-shaped substrate 1B, as illustrated in FIG. 3E and FIG. 4E, a process of forming the end face electrode 7 for connecting between the sulfidation detection conductor 3 and corresponding one of the back electrodes 5 at each end of the strip-shaped substrate 1B is performed. The end face electrode 7 is connected not only to the end of the sulfidation detection conductor 3 exposed from the protective layer 4, but also to an end face of the resistor 2 covered with the sulfidation detection conductor 3.

Next, after secondarily dividing the strip-shaped substrate 1B along the secondary division groove to obtain a plurality of chip-shaped substrates 1C, by forming electrolytic plating layers on each of these chip-shaped substrates 1C, a process of sequentially forming the intermediate electrode 8 formed of a Ni plating layer and the external electrode 9 formed of a Sn plating layer is performed. Thus, as illustrated in FIG. 3F and FIG. 4F, each of the electrode portions 6 including the end face electrode 7, the intermediate electrode 8, and the external electrode 9 is formed at both ends of the chip-shaped substrate 1C, whereby the sulfidation detection sensor 10 illustrated in FIG. 1 and FIG. 2 can be obtained.

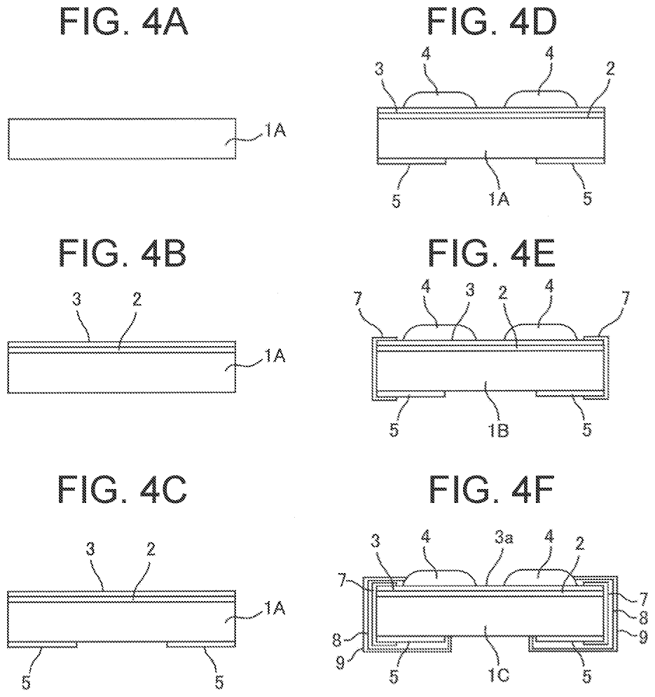

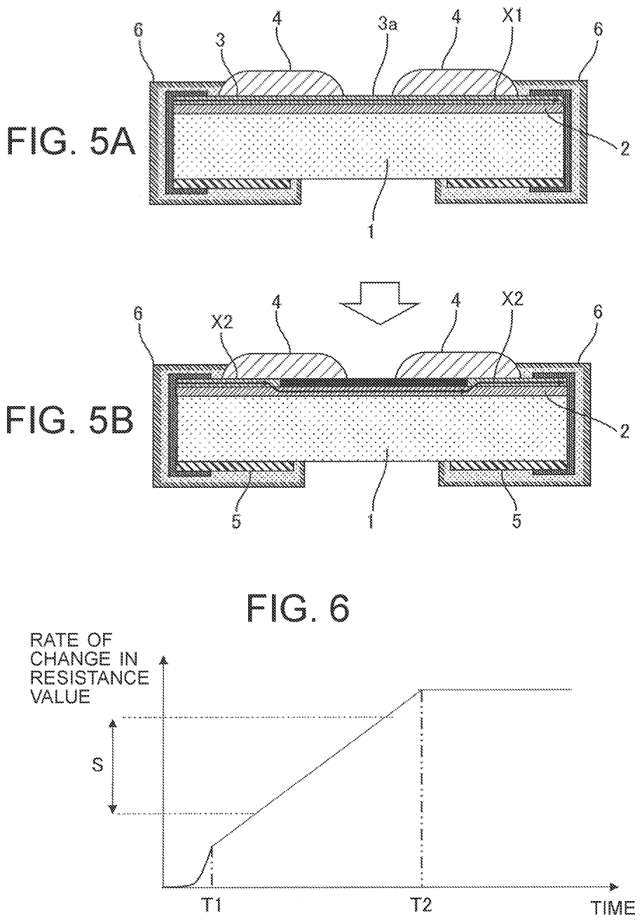

Each of FIG. 5A and FIG. 5B illustrates change in a current path when the sulfidation detection sensor 10 according to the present embodiment is disposed in a sulfide gas atmosphere, and FIG. 6 illustrates the relation between an elapsed time and a resistance value when the sulfidation detection sensor 10 is disposed in the sulfide gas atmosphere.

In the initial state before the sulfidation detection sensor 10 is exposed to the sulfide gas, the entire surface of the resistor 2 is covered with the sulfidation detection conductor 3, and both ends of the resistor 2 and those of the sulfidation detection conductor 3 are connected to the pair of electrode portions 6, respectively. Accordingly, as indicated by an arrow X1 in FIG. 5A, the current flowing between the pair of electrode portions flows in the sulfidation detection conductor 3 having a resistance value significantly smaller than that of the resistor 2.

When the sulfidation detection sensor 10 is disposed in an atmosphere containing the sulfide gas, the exposed portion 3a of the sulfidation detection conductor 3, which is exposed to the outside without being covered with the protective layer 4, comes into contact with the sulfide gas, whereby the exposed portion 3a of the sulfidation detection conductor 3 starts to get sulfurized with the elapse of time. Thereafter, the sulfidation proceeds to the inside of the sulfidation detection conductor 3 covered with the protective layer 4. This causes change in the current path in such a manner that, as indicated by an arrow X2 in FIG. 5B, it extends from a portion of one side of the sulfidation detection conductor 3, which has not been sulfurized, to a portion of the other side thereof, which has not been sulfurized, through the resistor 2. In accordance with this change in the current path, a resistance value between the pair of electrode portions changes as illustrated in FIG. 6. That is, the resistance value of the sulfidation detection sensor 10 gradually rises along a gentle curve to a time point (T1) at which the exposed portion 3a of the sulfidation detection conductor 3 starts to get sulfurized, then linearly rises as the sulfidation proceeds to the inside of the sulfidation detection conductor 3, and becomes constant (resistance value of the resistor 2) at a time point (T2) at which the entire sulfidation detection conductor 3 is sulfurized. Thus, within a threshold indicated by a symbol S in FIG. 6, the resistance value of the resistor 2 continuously changes in accordance with the degree of sulfidation of the sulfidation detection conductor 3, thereby enabling accurate and easy detection of the degree of sulfidation.

As described above, in the sulfidation detection sensor 10 according to the first embodiment, the pair of electrode portions 6 is always conductive to each other through the resistor 2 formed on the insulating substrate 1, and when the sulfidation detection sensor 10 is exposed in an atmosphere containing sulfide gas and thus sulfidation proceeds, the sulfidation detection conductor 3 provided on the resistor 2 starts to get sulfurized, from the exposed portion 3a exposed to the outside without being covered with the protective layer 4, and then toward the inside thereof covered with the protective layer 4. This causes change in the current path extending between the pair of electrode portions 6 in accordance with the degree of sulfidation of the sulfidation detection conductor 3. As a result, the resistance value of the resistor 2 continuously changes in accordance with the degree of sulfidation of the sulfidation detection conductor 3, thereby enabling accurate and easy detection of the degree of sulfidation.

Furthermore, in the sulfidation detection sensor 10 according to the first embodiment, since the resistor 2 and the sulfidation detection conductor 3 are metal films formed as thin films by sputtering or the like, it is possible to eliminate variations in the film thicknesses of the resistor 2 and sulfidation detection conductor 3, and thus enhance the accuracy of detection. Moreover, since the resistor 2 is a metal film of Ni—Cr formed as a thin film on the surface of the insulating substrate (alumina substrate) 1, it is possible to increase the bonding force between the resistor 2 and the alumina substrate 1 due to Cr in the metal film, and also, due to Ni therein, increase the adhesion between the resistor 2 and the sulfidation detection conductor 3 made of Ag, Cu, Ni or the like.

Note that using Cr can improve the sulfidation resistance of the metal film per se, however, increase in the content of Cr in the metal film mechanically weakens the metal film. Thus, it is preferable that the content of Cr in the metal film falls within a range of 40 to 60 wt %. Furthermore, since it is sufficient that the metal film made of Ni—Cr contains Ni—Cr as a main component, as long as the functions described above can be maintained, it may contain titanium (Ti), tungsten (W) or the like as appropriate so as to lower the thermal characteristics (TCR).

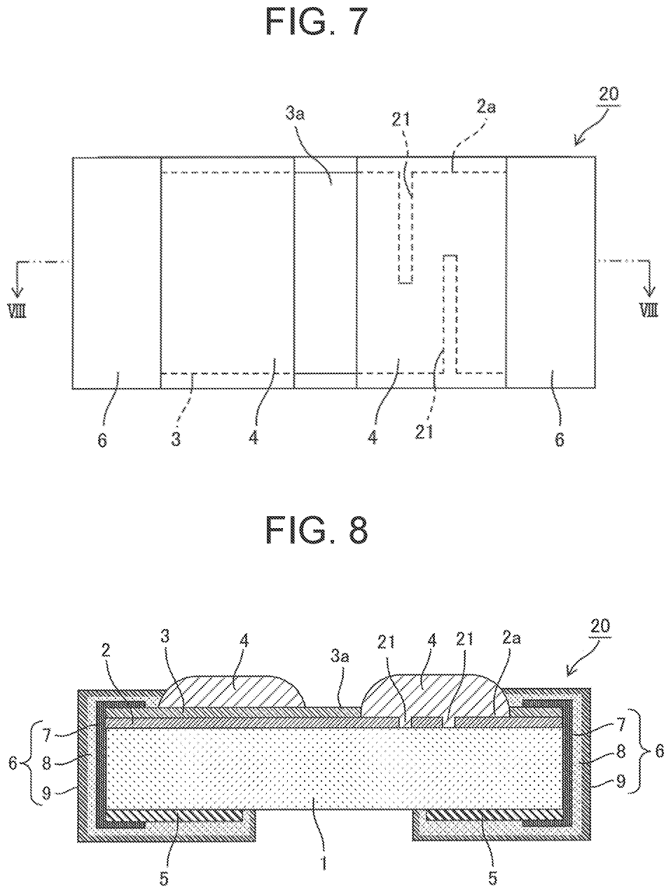

FIG. 7 is a plan view of a sulfidation detection sensor 20 according to a second embodiment of the present invention, and FIG. 8 is a cross-sectional view taken along the line VIII-VIII illustrated in FIG. 7. The portions corresponding to those illustrated in FIG. 1 and FIG. 2 are provided with the same reference signs.

As illustrated in FIG. 7 and FIG. 8, in the sulfidation detection sensor 20 according to the second embodiment, the resistor 2 includes an adjustment region 2a which is not covered with the sulfidation detection conductor 3. The adjustment region 2a is provided with a trimming groove 21 for adjusting a resistance value, and covered with the protective layer 4.

In the sulfidation detection sensor 20 designed as described above, forming the trimming groove 21 in the adjustment region 2a enables increase in an initial resistance value of the resistor 2, and also realizes the sulfidation detection sensor 20 which excels in the temperature characteristics (TCR). Note that the shape of the trimming groove 21 is not limited to I-cut shape as illustrated in FIG. 7 and FIG. 8, but may be other shapes such as L-cut shape. Moreover, the number of trimming grooves 21 to be provided is not limited to two as illustrated in FIG. 7 and FIG. 8, but may be appropriately increased or decreased.

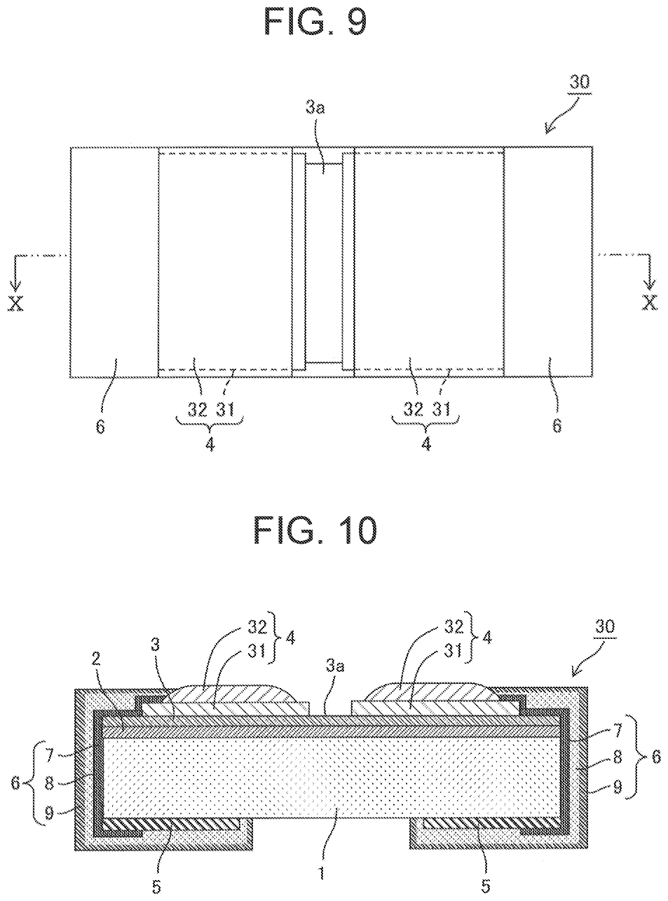

FIG. 9 is a plan view of a sulfidation detection sensor 30 according to a third embodiment of the present invention, and FIG. 10 is a cross-sectional view taken along the line X-X illustrated in FIG. 9. The portions corresponding to those illustrated in FIG. 1 and FIG. 2 are provided with the same reference signs.

As illustrated in FIG. 9 and FIG. 10, in the sulfidation detection sensor 30 according to the third embodiment, the protective layer 4 is composed of an undercoat layer 31 made of a glass material formed on the sulfidation detection conductor 3 and an overcoat layer 32 made of a resin material formed on the undercoat layer 31. Each of the electrode portions 6 covers the ends of the sulfidation detection conductor 3 and adheres closely to the overcoat layer 32.

In the sulfidation detection sensor 30 designed as described above, providing the overcoat layer 32 made of a resin material in the protective layer 4 improves the adhesion between the overcoat layer 32 and the electrode portions 6, so that it is possible to suppress sulfidation of each end of the sulfidation detection conductor 3 covered with the end face electrode 7 of the electrode portion 6. However, since the resin material is permeable to gas, forming the entire protective layer 4 with a resin material may cause, due to sulfide gas which has permeated through the protective layer 4, sulfidation of the sulfidation detection conductor 3 positioned directly below the protective layer 4. In this regard, forming the undercoat layer 31 made of a glass material, which is impermeable to the sulfide gas, under the overcoat layer 32 made of a resin material, prevents the sulfidation detection conductor 3 positioned directly below the protective layer 4 from reacting with the sulfide gas which has permeated through the overcoat layer 32 and thus getting sulfurized.

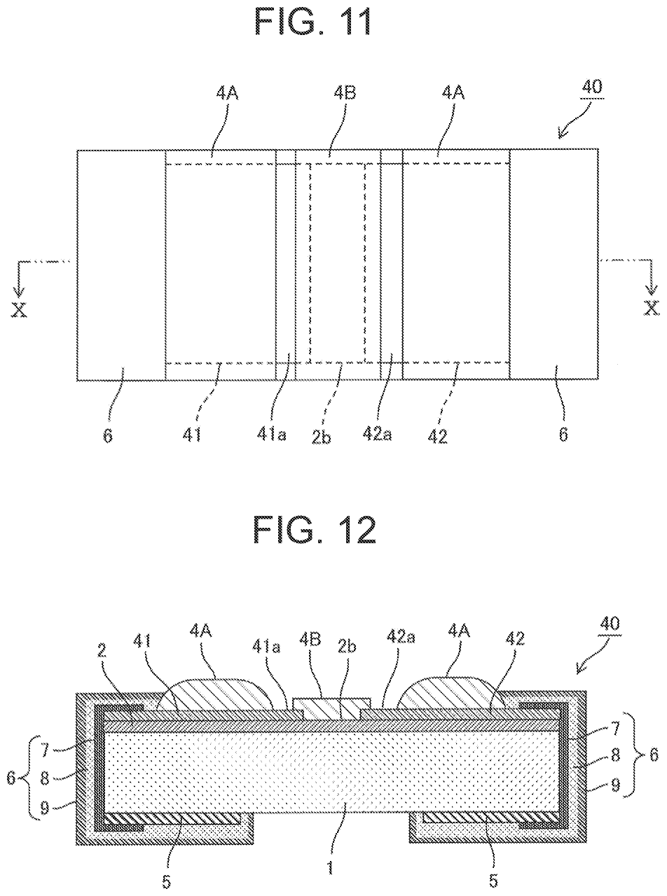

FIG. 11 is a plan view of a sulfidation detection sensor 40 according to a fourth embodiment of the present invention, and FIG. 12 is a cross-sectional view taken along the line XII-XII illustrated in FIG. 11. The portions corresponding to those illustrated in FIG. 1 and FIG. 2 are provided with the same reference signs.

As illustrated in FIG. 11 and FIG. 12, in the sulfidation detection sensor 40 according to the fourth embodiment, the resistor 2 includes an exposed region 2b at the central portion in the longitudinal direction, and a first sulfidation detection conductor 41 and a second sulfidation detection conductor 42 are formed at two positions of the resistor 2 so as to interpose the exposed region 2b therebetween. Each of the first sulfidation detection conductor 41 and the second sulfidation detection conductor 42 is made of a metal film formed as a thin film by sputtering, vapor deposition or the like on the surface of the resistor 2, however, the kind of material of the metal film of the first sulfidation detection conductor 41 is different from that of the second sulfidation detection conductor 42. For example, the first sulfidation detection conductor 41 is a metal film of Ni while the second sulfidation detection conductor 42 is a metal film of Ag.

At a central portion of the first sulfidation detection conductor 41, a protective layer 4A which is impermeable to sulfide gas is formed, and the inner end of the first sulfidation detection conductor 41 is an exposed portion 41a which is not covered with the protective layer 4A and exposed to the outside. Similarly, at a central portion of the second sulfidation detection conductor 42, another protective layer 4A which is impermeable to sulfide gas is formed, and the inner end of the second sulfidation detection conductor 42 is an exposed portion 42a which is not covered with the protective layer 4A and exposed to the outside. In addition, an intermediate protective layer 4B which is impermeable to sulfide gas is also formed at the exposed region 2b of the resistor 2, and the exposed portion 41a of the first sulfidation detection conductor 41 and the exposed portion 42a of the second sulfidation detection conductor 42 are disposed at opposing positions interposing the intermediate protective layer 4B therebetween.

In the sulfidation detection sensor 40 designed as described above, the first sulfidation detection conductor 41 and the second sulfidation detection conductor 42, which are made of different materials with different selectivity of gas, are formed on the resistor 2, and the first sulfidation detection conductor 41 and the second sulfidation detection conductor 42 include the exposed portions 41a, 42a, respectively. This enables reliable detection of the degree of sulfidation regardless of the type of sulfide gas contained in an atmosphere during use.

That is, the reactivity of sulfide gas differs depending on the type of metal forming the sulfidation detection conductor, for example, silver (Ag) easily reacts with hydrogen sulfide (H₂S) but hardly reacts with sulfur dioxide (SO₂) while nickel (Ni) easily reacts with sulfur dioxide (SO₂) but hardly reacts with hydrogen sulfide (H₂S), and accordingly, in a gas atmosphere containing sulfur dioxide, the exposed portion 41a of the first sulfidation detection conductor 41 made of Ni starts to get sulfurized while in a gas atmosphere containing hydrogen sulfide, the exposed portion 42a of the second sulfidation detection conductor 42 made of Ag starts to get sulfurized. As a result, it is possible to realize the sulfidation detection sensor 40 that is applicable for different types of sulfide gases.

Note that, since copper (Cu) is a material that is easy to react with both hydrogen sulfide (H₂S) and sulfur dioxide (SO₂), solely using copper (Cu) can realize a sulfidation detection sensor which is applicable for multiple types. However, like the combination of Ag material and Ni material as described above, combining copper (Cu) with a material with different selectivity of gas and high responsibility to the target sulfide gas can improve the accuracy of detection more than the case of forming a sulfidation detection body solely using copper (Cu).

Furthermore, in the sulfidation detection sensor 40 according to the fourth embodiment, the resistor 2 includes the exposed region 2b which is not covered with the first sulfidation detection conductor 41 and the second sulfidation detection conductor 42, the intermediate protective layer 4B is formed so as to cover the exposed region 2b, and the exposed portion 41a of the first sulfidation detection conductor 41 and the exposed portion 42a of the second sulfidation detection conductor 42 are provided at the opposing positions interposing the intermediate protective layer 4B therebetween. This makes it easy to form, by sputtering (mask sputtering) Ni and Ag from above a mask onto the surface of the resistor 2, the first sulfidation detection conductor 41 and the second sulfidation detection conductor 42 which are made of different materials.

In each of the embodiments above, an example in which the resistor 2 and the sulfidation detection conductor 3 (41, 42) are metal films formed as thin films by sputtering or the like has been described. On the other hand, the resistor and the sulfidation detection conductor may be formed with metal glaze thick films. For example, the resistor may be formed by screen-printing an Ag—Pd (50%) paste and then drying and sintering the paste, and the sulfidation detection conductor may be formed by screen-printing a Cu paste or Ag paste and then drying and sintering the paste.

### REFERENCE SIGNS LIST

1 insulating substrate
1A large-sized substrate
1B strip-shaped substrate
1C chip-shaped substrates
2 resistor
2a adjustment region
2b exposed region
3 sulfidation detection conductor
3a exposed portion
4, 4A protective layer
4B intermediate protective layer
5 back electrode
6 electrode portion
7 end face electrode
8 intermediate electrode
9 external electrode
10, 20, 30, 40 sulfidation detection sensor
21 trimming groove
31 undercoat layer
32 overcoat layer
41 first sulfidation detection conductor
41a exposed portion
42 second sulfidation detection conductor
42a exposed portion

1. A sulfidation detection sensor, comprising:
a rectangular parallelepiped insulating substrate;
a resistor that is provided on a main surface of the insulating substrate;
a sulfidation detection conductor that is provided on the resistor and to be sulfurized by sulfide gas;
a protective layer that is impermeable to the sulfide gas, and provided so as to cover a portion of the sulfidation detection conductor; and
a pair of electrode portions that is provided at both ends of the insulating substrate, respectively, and connected to the resistor and the sulfidation detection conductor,
wherein
the sulfidation detection conductor is made of metal having a resistance value smaller than that of the resistor, and includes an exposed portion exposed to an outside without being covered with the protective layer.

2. The sulfidation detection sensor according to claim 1, wherein
the resistor includes an adjustment region which is not covered with the sulfidation detection conductor,
the adjustment region is provided with a trimming groove for adjusting a resistance value, and
the adjustment region is covered with a part of the protective layer.

3. The sulfidation detection sensor according to claim 1, wherein
the sulfidation detection conductor is composed of a first sulfidation detection conductor and a second sulfidation detection conductor which are made of different materials with different selectivity of gas, and
each of the first sulfidation detection conductor and the second sulfidation detection conductor includes the exposed portion.

4. The sulfidation detection sensor according to claim 3, wherein
the resistor includes an exposed region which is not covered with the first sulfidation detection conductor and the second sulfidation detection conductor,
an intermediate protective layer is formed on the exposed region, and
the exposed portion of the first sulfidation detection conductor and the exposed portion of the second sulfidation detection conductor are disposed at positions interposing the intermediate protective layer therebetween.

5. The sulfidation detection sensor according to claim 1, wherein
each of the resistor and the sulfidation detection conductor is made of a metal film formed as a thin film.

6. The sulfidation detection sensor according to claim 5, wherein
the insulating substrate is made of an alumina substrate, and
the resistor is a metal film of Ni—Cr formed by sputtering on a surface of the alumina substrate.

7. The sulfidation detection sensor according to claim 1, wherein
the protective layer is composed of an undercoat layer made of a glass material formed on the sulfidation detection conductor and an overcoat layer made of a resin material formed on the undercoat layer, and
each of the electrode portions covers an end of the sulfidation detection conductor and adheres closely to the overcoat layer.
